(19) United States
(12) Patent Application Publication
(10) Pub. No.: US 2004/0067222 A1
(43) Pub. Date: Apr. 8, 2004

(54) Regulation of glucocorticoid concentration

(76) Inventors: Brian Robert Walker, Edinburgh (GB); Jonathan Robert Seckl, Edinburgh (GB); John Savill, Edinburgh (GB); Jamie Gilmour, Edinburgh (GB); Karen Chapman, Edinburgh (GB)

Correspondence Address: FROMMER LAWRENCE & HAUG, 745 FIFTH AVENUE- 10TH FL., NEW YORK, NY, 10151, US

(21) Appl. No.: 10/622,653

(22) Filed: Jul. 18, 2003

Related U.S. Application Data
(63) Continuation-in-part of application No. PCT/GB02/00255, filed on Jan. 21, 2002.

(30) Foreign Application Priority Data
Jan. 19, 2001 (GB) 0101447.1

(51) Int. Cl. A61K 48/00; A61K 31/573; C12N 5/08

(52) U.S. Cl. 424/93.21; 514/179; 435/372

(57) ABSTRACT

The invention relates to use of a modulator of glucocorticoid metabolism, or the administration of inactive glucocorticoids, for the potentiation of a successful resolution of an inflammatory response in a mammal.

### REFERENCE TO RELATED APPLICATIONS

This application is a continuation-in-part of International Application PCT/GB02/00255 filed on Jan. 21, 2002 and published as WO 02/056891 on Jul. 25, 2002, which application claims priority from Great Britain Application 0101447.1 filed Jan. 19, 2001.

Each of the foregoing applications, and each document cited or referenced in each of the foregoing applications, including during the prosecution of each of the foregoing applications and (“application cited documents”), and any manufacturer's instructions or catalogues for any products cited or mentioned in each of the foregoing applications and articles and in any of the application cited documents, are hereby incorporated herein by reference. Furthermore, all documents cited in this text, and all documents cited or referenced in documents cited in this text, and any manufacturer's instructions or catalogues for any products cited or mentioned in this text or in any document hereby incorporated into this text, are hereby incorporated herein by reference. Documents incorporated by reference into this text or any teachings therein may be used in the practice of this invention. Documents incorporated by reference into this text are not admitted to be prior art.

The present invention relates to the regulation of glucocorticoid levels. In particular, the invention relates to the regulation of intracellular glucocorticoid levels in macrophages to enhance the successful resolution of the inflammatory response mediated by such cells.

Glucocorticoids such as cortisol have a number of diverse effects on different body tissues. Our International Patent Application WO 90/04399 was concerned with the problem that therapeutically administered cortisol tends to be converted in the body to inactive cortisone by 11β-hydroxysteroid dehydrogenase enzymes (11β-HSDs). Our earlier invention provided for the potentiation of cortisol action by the administration of an inhibitor of the 11β-dehydrogenase activity of these enzymes. The 11β-HSD enzyme addressed in WO 90/04399 is the 11β-HSD2 enzyme, which is exclusively a dehydrogenase for endogenous glucocorticoids, converting cortisol to cortisone.

It is also known that the reverse reaction, converting inactive cortisone to active cortisol, is accomplished in certain organs by 11β-reductase activity of the 11β-HSD1 enzyme. This activity is also known as corticosteroid 11β-reductase, cortisone 11β-reductase, or corticosteroid 11β-oxidoreductase.

Expression of 11β-HSD1 in a range of cell lines encodes either a bi-directional enzyme [Agarwal A K, Monder C, Eckstein B & White P C J Biol Chem 264, 18939-18943 (1989); Agarwal A K, Tusie-Luna M-T, Monder C & White P C Mol Endocrinol 4, 1827-1832 (1990)] or a predominant 11β-reductase [Duperrex H, Kenouch S, Gaeggleler H P, et al. Endocrinology 132, 612-619 (1993); Jamieson P M, Chapman K E, Edwards C R W & Secki J R. Endocrinology 136, 4754-4761 (1995)] which, far from inactivating glucocorticoids, regenerates active 11β-hydroxysteroid from otherwise inert 11-keto steroid. 11β-reductase activity, best observed in intact cells, activates 11-keto steroid to alter target gene transcription and differentiated cell function [Duperrex H, Kenouch S, Gaeggleler H P, et al. Endocrinology 132, 612-619 (1993); Low S C, Chapman K E, Edwards C R W & Seckl J R Journal of Molecular Endocrinology 13, 167-174 (1994)]. 11β-HSD1 and 11β-HSD2 are the products of different genes and share only 20% amino acid homology [Agarwal A K, Mune T, Monder C & White P C (1994) J Biol Chem 269, 25959-25962 (1994); Albiston A L, Obeyesekere V R, Smith R E & Krozowski Z S Mol Cell Endocrinol 105, R11-R17 (1994)]. In our International patent application WO97/07789, the contents of which and documents referenced therein being incorporated herein by reference, we discuss the inhibition of 11β-reductase activity in vivo, and the treatment of many of the deleterious effects of glucocorticoid excess.

Cortisol promotes hepatic gluconeogenesis by several mechanisms, including antagonism of the effects of insulin on glucose transport, and interactions with insulin and glucose in the regulation of several enzymes which control glycolysis and gluconeogenesis. These include glucokinase, 6-phosphofructokinase, pyruvate kinase, phosphoenolpyruvate carboxykinase (PEPCK), and glucose-6-phosphatase. Inhibiting production of cortisol from cortisone in the liver therefore enhances hepatic glucose uptake and inhibits hepatic glucose production by several mechanisms. Moreover, the influence of inhibiting 11β-reductase activity in the liver of patients with insulin resistance or glucose intolerance can be greater than in healthy subjects because in insulin resistance or deficiency the influence of cortisol on PEPCK has been shown to be greater; obese patients secrete more cortisol; insulin resistant patients are more sensitive to glucocorticoids; and insulin down-regulates 11β-HSD1 expression so that 11β-reductase activity can be enhanced in conditions of insulin resistance or deficiency.

Our International patent application WO97/07789 also shows that 11β-HSD1 is expressed in rat adipose tissue and in adipocyte cell lines in culture, where it converts 11-dehydrocorticosterone to corticosterone (the rat equivalents of human cortisone and cortisol, respectively). This suggests that similar 11β-reductase activity will be observed in human adipose tissue, with the result that inhibition of the enzyme will result in alleviation of the effects of insulin resistance in adipose tissue in humans. This would lead to greater tissue utilisation of glucose and fatty acids, thus reducing circulating levels. The invention therefore provides, in a further aspect, the use of an inhibitor of 11β-reductase in the manufacture of a medicament for increasing insulin sensitivity in adipose tissue.

It is also known that glucocorticoid excess potentiates the action of certain neurotoxins, which leads to neuronal dysfunction and loss. We have studied the interconversion between 11-dehydrocorticosterone and corticosterone in rat hippocampal cultures, and have found (surprisingly in view of the damaging effects of glucocorticoids) that 11β-reductase activity dominates over 11β-dehydrogenase activity in intact hippocampal cells. The reason for this activity is unknown, but this result indicates that glucocorticoid excess can be controlled in hippocampal cells (and by extension in the nervous system in general) by use of an 11β-reductase inhibitor, and the invention therefore provides in an alternative aspect the use of an inhibitor of 11β-reductase in the manufacture of a medicament for the prevention or reduction of neuronal dysfunction and loss due to glucocorticoid potentiated neurotoxicity. It is also possible that glucocorticoids are involved in the cognitive impairment of ageing with or without neuronal loss and also in dendritic attenuation. Furthermore, glucocorticoids have been implicated in the neuronal dysfunction of major depression. Thus an inhibitor of 11β-reductase could also be of value in these conditions.

Our earlier International patent application, therefore, provides that the beneficial effects of inhibitors of 11β-reductase are many and diverse, and it is envisaged that in many cases a combined activity will be demonstrated, tending to relieve the effects of endogenous glucocorticoids in diabetes mellitus, obesity (including centripetal obesity), neuronal loss and the cognitive impairment of old age. However, the effects of glucocorticoids on macrophages are not described.

The system inflammatory diseases of the lungs, joints, kidneys and gut exert a heavy toll upon society. Current treatments for inflammatory disorders have concentrated on blocking initiation and amplification mechanisms of inflammation, in other words on preventing or arresting the inflammatory process using anti-inflammatory treatments. Unfortunately, these do not prevent progression of persistent inflammation to scarring and loss of organ function.

A growing body of data now points to apoptosis or programmed cell death being a key mechanism for safe removal of leukocytes from inflamed sites. Thus, apoptosis in the leukocyte packages the leukocyte and its noxious contents for safe uptake and degradation by phagocytes. Furthermore, there are data (e.g. Taylor et al., J Exp Med. Aug. 7, 2000;192(3):359-66) which establish effective macrophage clearance of apoptotic cells as a key pathogenic factor in disorders characterised by persistent inflammation and autoimmunity, such as systemic lupus erythematosus.

Therefore, means to enhance the clearance of apoptotic leukocytes from inflamed sites are required. Furthermore, this could be a generally important approach toward promoting resolution of inflammation, even where intrinsic defects in clearance may not be present.

### SUMMARY OF THE INVENTION

The present invention provides a new approach to the treatment of inflammatory conditions, in which inflammation is promoted rather than prevented. In accordance with the invention, inflammation is promoted to its resolution, such that the natural biological benefits of the inflammatory process can be exploited. We have now determined that glucocorticoid activity in macrophages stimulates the termination of the inflammatory response to reach a successful outcome. Our studies indicate that glucocorticoid (GC) treatment specifically enhances the non-inflammatory phagocytosis of apoptotic neutrophils (PMN) by macrophages. Moreover, the potentiation of 11β-HSD1 activity in macrophages increases intracellular glucocorticoid levels to achieve the same beneficial effects.

According to a first aspect of the present invention, therefore, we provide the use of a modulator of glucocorticoid metabolism in the manufacture of a composition for the potentiation of a successful resolution of an inflammatory response in a mammal.

The modulator in accordance with the invention preferably increases the intracellular concentration of active glucocorticoid in phagocytes active in the phagocytosis of apoptotic leukocytes. Advantageously, the phagocytes are macrophages. Preferably, therefore, the modulator of glucocorticoid metabolism is selectively delivered to phagocytic cells, ideally at the site of inflammation. For example, it is selectively delivered to macrophages.

In an advantageous embodiment, this can be achieved by increasing the intracellular activity of 11β-HSD reductase, either by administering 11β-HSD enzyme or by administering a modulator of 11β-HSD reductase activity.

In a second embodiment, the invention provides an engineered macrophage in which endogenous active glucocorticoid levels have been increased. This can be achieved, for example, by genetically engineering the macrophages, such as to increase 11β-HSD activity therein. The macrophages according to the invention are useful in the treatment of conditions in which inflammatory responses are advantageously managed to a successful resolution.

Engineered macrophages can be delivered to the site of inflammation in an individual. Macrophages naturally home to inflamed tissues when introduced into a subject.

Preferably, the 11β-HSD enzyme is 11β-HSD 1.

Both active glucocorticoid and inactive 11-keto steroids (such as 11-dehydrocorticosteroids) can also be used in accordance with the present invention. Particularly inactive precursors of glucocorticoid, such as 11-dehydroxycorticosterone, which are converted to active forms by 11β-HSD1 or equivalent enzymes, are useful as substrates which can be administered to sites of inflammation and converted in situ by macrophages to active glucocorticoid. Glucocorticoid and/or inert precursor can be administered in combination with a modulator of glucocorticoid metabolism, or with an engineered macrophage in accordance with the above aspects of the invention.

Reactivation by 11β-HSD type 2 is a further option in that this enzyme has a considerably more restricted distribution, mainly being located in mineralocorticoid sensitive organs. 9α-Fluorinated steroids are reactivated by this enzyme. In the case of 11-dehydrodexamethasone, this would yield an active glucocorticoid. Thus, the invention provides for the delivery of active 11β-HSD 2 to macrophages, and subsequent treatment with one or more 9α-Fluorinated steroids. Advantageously, the macrophages are engineered macrophages expressing 11β-HSD 2.

Thus, the invention provides at least two of a glucocorticoid and/or inactive precursor steroid and/or an engineered macrophage and/or a modulator of glucocorticoid metabolism, as described above, for separate, simultaneous separate or sequential use in the potentiation of a successful resolution of the inflammatory response in a mammal.

Preferably, the agent(s) according to the invention are targeted to phagocytic cells, such as macrophages.

Moreover, the invention provides a pharmaceutical composition comprising one or more of a glucocorticoid and/or inactive precursor steroid and/or an engineered macrophage and/or a modulator of glucocorticoid metabolism, as described above.

In a further aspect, there is provided a method of potentiating a successful inflammatory response in a mammal, comprising administering to a mammal in need thereof a composition comprising a glucocorticoid and/or inactive precursor steroid and/or an engineered macrophage and/or a modulator of glucocorticoid metabolism, as described above.

### BRIEF DESCRIPTION OF THE FIGURES

FIG. 1 is a photomicrograph showing macrophage phagocytosis in the presence and absence of dexamethasone.

FIG. 2 shows (a) RT-PCR of transcripts obtained from kidney, liver and macrophages; (b) the conversion of inactive precursor 11-dehydrocorticosterone (A) to active glucocorticoid (B); and (c) inhibition thereof with carbenoxolone.

FIG. 3 is a bar graph representing the levels of macrophage phagocytosis in the presence of active glucocorticoid (B) and inactive (A) precursor corticosteroid, and the effect of carbenoxolone treatment.

FIG. 4 is a bar graph representing the effects of active glucocorticoid (AGC) and inactive precursor corticosteroid (IGC) on macrophage phagocytosis in macrophages derived from wild-type and 11β-HSD 1^{-/-} mice.

FIG. 5 shows the developmental regulation of glucocorticoid-activating activity in the monocyte/macrophage lineage.

FIG. 6 shows the developmental regulation of glucocorticoid-activating activity in the monocyte/macrophage lineage, linked to 11β-HSD 1 expression in macrophages.

FIG. 7 shows the developmental regulation of phagocytosis in the monocyte/macrophage lineage, linked to 11β-HSD 1 expression in macrophages.

FIG. 8 shows the induction of glucocorticoid activating activity in macrophages by IL-4 treatment.

FIG. 9 shows the glucocorticoid-activating activity of macrophages during and after a macrophage phagocytosis experiment.

FIG. 10 shows the glucocorticoid-activating activity of macrophages during and after in vivo peritonitis induction in mice by thioglycollate treatment.

### DETAILED DESCRIPTION OF THE INVENTION

Definitions

Unless defined otherwise, all technical and scientific terms used herein have the same meaning as commonly understood by one of ordinary skill in the art (e.g., in cell culture, molecular genetics, nucleic acid chemistry, hybridisation techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc. which are incorporated herein by reference) and chemical methods.

A “modulator” is an agent which increases or decreases a level of that which is modulated. For example, it may be an agent which increases or decreases the abundance, effect, activity, concentration or bioavailability of a modulated substance, which may be a gene product or a compound such as a glucocorticoid.

A “glucocorticoid” is any member of the family of steroid hormones (both natural and synthetic) that bind glucocorticoid receptors and thereby influence gene transcription. Their actions include promoting gluconeogenesis and the formation of glycogen at the expense of lipid and protein synthesis, and important anti-inflammatory activity. Exemplary glucocorticoids include hydrocortisone (cortisol), prednisolone, dexamethasone and betamethasone. Glucocorticoids may be formed from inactive precursor corticosteroids by the 11β-reductase activity of 11β-HSD1 or 11β-HSD 2, including cortisol from cortisone, and prednisolone from prednisone.

A “modulator of glucocorticoid metabolism” is any compound, substance or treatment which upregulates or downregulates the activity (such as by increasing the abundance, effect, concentration or bioavailability) of glucocorticoid in a cell. Advantageously, the cell is a macrophage. The activity of the glucocorticoid is preferably increased in the macrophage, for example by increasing the biosynthesis of active glucocorticoid or the conversion of inactive forms of glucocorticoid to active glucocorticoid. Thus, for example, the modulator can increase the levels of 11β-HSD enzymes in the macrophage, which is shown in the present invention to lead to advantageous effects in the phagocytosis of apoptotic cells and thus the successful resolution of an inflammatory response. The modulator can therefore be, for example, exogenously administered 11β-HSD enzyme itself, or a nucleic acid encoding 11β-HSD1 or 11β-HSD 2 which is delivered to the cell such that it can be expressed therein to produce increased levels of 11β-HSD1 or 11β-HSD 2.

An “inflammatory response” is typically a response to injury or infection/disease which involves inflammation of tissues. Acute inflammation is dominated by vascular changes and by neutrophil leukocytes in the early stages, mononuclear phagocytes in the later stages. Leukocytes adhere locally and emigrate into the tissue between the endothelial cells lining of the post-capillary venules. Plasma exudation from vessels may lead to tissue swelling, but the early vascular changes are independent of and not essential for the later cellular response. In chronic inflammation, where the stimulus is persistent, the characteristic cells present are macrophages and lymphocytes. Inflammation is generally beneficial, and assists the return of homeostasis after injury or disease.

A “successful resolution of the inflammatory response” is an inflammatory response in which the desired outcome to prevent the occurrence of chronicity (phagocytosis of apoptotic cells) is increased or otherwise potentiated. It is not synonymous with anti-inflammatory treatment. The invention potentiates, that is better achieves the benefits of, the natural inflammatory response; it does not avoid an inflammatory response, but assists its purpose and aids its prompt resolution.

A “macrophage” is a relatively long-lived phagocytic cell of mammalian tissues, derived from blood monocytes. Main types of macrophage include peritoneal and alveolar macrophages, tissue macrophages (histiocytes), Kuppfer cells of the liver, and osteoclasts. In response to foreign materials or disease macrophages become stimulated or activated. Macrophages play an important role in killing of some bacteria, protozoa, and tumour cells, release substances that stimulate other cells of the immune system, and are involved in antigen presentation. Macrophages may also be further differentiated cells found within chronic inflammatory lesions, such as epithelioid cells or fused cells which form foreign body giant cells or Langerhans' giant cells.

An “engineered” macrophage is a macrophage which has been modified in order to increase the levels of active glucocorticoid therein. This can be achieved, in a preferred embodiment, by engineering the macrophage to express increased levels of 11β-HSD1 or 11β-HSD 2. This enzyme acts as a reductase in the macrophage and increases the conversion of inactive glucocorticoid to its active form. Methods for engineering macrophages to produce elevated levels of 11β-HSD1 or 11β-HSD 2 are known to those skilled in the art and further described below.

Glucocorticoids

Glucocorticoids are a group of adrenocortical steroid hormones whose metabolic effects include stimulation of gluconeogenesis, increased catabolism of proteins, and mobilisation of free fatty acids; they are also know to be potent inhibitors of the inflammatory response (allergic response). The vast majority of glucocorticoid activity in most mammals is from cortisol, also known as hydrocortisone. Corticosterone is the major glucocorticoid in rodents. Synthetic glucocorticoids are also known, such as dexamethasone. Cortisol binds to the glucocorticoid receptor in the cytoplasm and the hormone-receptor complex is then translocated into the nucleus, where it binds to its DNA response elements and modulates transcription of relevant genes.

Glucocorticoid receptors are universally present and as a consequence, these steroid hormones have a huge number of effects on physiological systems. The best known and studied effects of glucocorticoids are on carbohydrate metabolism and immune function. Indeed, the name glucocorticoid derives from early observations that these hormones were involved in glucose metabolism. In the fasting state, cortisol stimulates several processes that collectively serve to increase and maintain normal concentrations of glucose in blood.

Glucocorticoids are known to have potent anti-inflammatory and immunosuppressive properties. This is particularly evident when they administered at pharmacological doses, but also is important in normal immune responses. As a consequence, glucocorticoids are widely used as drugs to treat chronic (unnecessarily persistent) inflammatory conditions such as arthritis, nephritis, asthma or dermatitis, and as adjunction therapy for conditions such as autoimmune diseases.

Some of the steroid drugs for topical administration for anti-inflammatory purposes include Betamethasone (Diprolene® cream), Clobetasol (Temovate®), Desonide (Desowen®), Fluocinolone (Derma-Smoothe/FS®), Fluocinonide (Lidex®), Hydrocortisone (Anusol®, Cortaid®, Hydrocortone®), Mometasone (Elocon®) and Triamcinolone (Aristocort®, Knalog®). It is currently believed that the anti-inflammatory properties of glucocorticoids are due to their ability to regulate pro-inflammatory genes, or modulate cellular apoptosis.

Glucocorticoids circulate in inactive forms, which are reduced to active compounds at the site of action. 9-α-Fluorinated 11-dehydrocorticosteroids like 11-dehydro-dexamethasone (DH-D) are rapidly activated by 11β-reductase activity of 11β-HSD 2 to the active dexamethasone (D). 11-keto steroids such as cortisone are reduced to 11-hydroxy compounds such as cortisol by 11β-HSD 1. Similarly, prednisone is reduced to prednisolone. Moreover, hepatic 11β-HSD1 is known to reduce cortisone to cortisol in the liver. Thus, in the context of the present invention, an active glucocorticoid is the reduced form, such as cortisol or dexamethasone or prednisolone; and inactive glucocorticoid is, for example, cortisone or 11-dehydro-dexamethasone or prednisone.

Modulators of Glucocorticoid Metabolism

In a preferred embodiment, such modulators are enzymes which catalyse the conversion of inactive glucocorticoids to active glucocorticoids. Thus, the invention is particularly concerned with 11β-HSD reductase enzymes. For example, the human 11β-HSD2 enzyme is known and details thereof can be found at GenBank Accession No. M76661.1 GI:179469. The sequence of 11β-HSD1 can be found at Accession no. NM_{—}005525.1 GI:5031764. Modulators of the activity of such enzymes are also to be considered modulators of glucocorticoid metabolism; thus, compounds such as carbenoxolone, which inhibits 11β-HSD1, are encompassed by the invention, as are potentiators of 11β-HSD1 activity. See Monder and White, (1993) Vitamins and Hormones 47:187; especially Table IV thereof.

11β-HSD enzymes may themselves be modulated, for example by regulation of their induction in the macrophage or by other means. For example, cytokines such as IL-4 are capable of inducing 11β-HSD 1 expression, as shown herein; IL-4 itself or small molecule mimics or analogues thereof may be administered to macrophages through selective uptake, for example by cationic liposomes, as described below. Synthetic IL-4 analogues are known in the art, for example as described in Dominigues et al., Nat Struct Biol 1999 July;6(7):652-6. Using computer-aided molecular modelling, the putative IL-4 motif for binding to IL-4R (receptor) was transferred stepwise to a selected scaffold molecule that was the leucine-zipper domain of the yeast transcription factor GCN4. The resulting molecules bound IL-4R with affinities ranging from 2 mM to 5 M, depending on stability and fraction of the IL-4 binding motif incorporated.

Mimetics may also be non-peptidyl mimetics. Nonpeptidyl mimetics may be derived from natural sources or combinatorial libraries. For example, in a screen for small molecules that activate the human insulin receptor tyrosine kinase, a nonpeptidyl fungal metabolite (L-783,281) was identified that acted as an insulin mimetic in several biochemical and cellular assays. The compound was selective for insulin receptor, and oral administration of L-783,281 to two mouse models of diabetes resulted in significant lowering in blood glucose levels suggesting the feasibility of discovering novel insulin receptor activators that may lead to new therapies for diabetes (Zhang et al. Science May 7, 1999;284(5416):974-7). In chemical synthesis, a manganese(II) complex with a bis(cyclohexylpyridine)-substituted macrocyclic ligand (M40403) was designed to be a functional mimic of the superoxide dismutase (SOD) enzymes that normally remove radicals associated with many human diseases (Salvemini et al., Science Oct. 8, 1999;286(5438):304-6). Mimics of this nonpeptidyl nature may result in better clinical therapies for diseases mediated by superoxide radicals. Also in the field of blood coagulation, breakthroughs in oligosaccharide chemistry made possible the total synthesis of the pentasaccharide antithrombin-binding site of heparin, and Petitou et al., Nature Apr. 1, 1999;398(6726):417-22) reported a heparin mimetic that is sulphated oligosaccharides and without side effects such as heparin-induced thrombocytopaenia (HIT) and haemorrhages in heparinotherapy. A review of protein mimic design and selection may be found in Cochran, Chemistry and Biology (2000) 7:R85-R94.

Delivery to Macrophages

In a preferred embodiment, the present invention encompasses the delivery of modulators of glucocorticoid metabolism, including nucleic acids, polypeptides, chemical compounds and active or inactive glucocorticoids themselves, to macrophages. Techniques for delivery of drugs, nucleic acids and other agents to macrophages are known in the art. Many techniques use modified liposomes or nanoparticles, often carrying carbohydrate groups which are recognised and assimilated by macrophages. See, for example, Sihorkar V, Vyas S P, J Pharm Pharm Sci 2001 May-August;4(2):138-58; Moghimi et al., Pharmacol Rev. 2001 June;53(2):283-318; Couvreur P. & Vauthier C. (1994) In: Drug absorption enhancement. Concepts and limitations. Ed. A(Bert)GDboer. Harwood Academic Publishers. Leiden, Amsterdam; and international patent application WO 97/45442. Alvarez et al., Biotechnol. Appl. Biochem. (1998) 27, 139-143, describe the use of cross-linked erythrocytes to deliver pharmacological agents to macrophages.

Preferably, cationic liposomes are used to deliver the agent of choice to the macrophage. Suitable liposomes for use in the present invention are commercially available. DOTMA liposomes, for example, are available under the trademark Lipofectin from Bethesda Research Labs, Gaithersburg, Md. Alternatively, liposomes can be prepared from readily-available or freshly synthesised starting materials of the type previously described in the literature, see, e.g., P. Feigner, et al., Proc. Nat'l Acad. Sci. USA 84:7413-7417. Similar methods can be used to prepare liposomes from other cationic lipid materials.

Liposomes are selectively taken up by macrophages at sites of inflammation, making them the ideal vehicle for delivery of glucocorticoids and/or nucleic acids according to the present invention.

Moreover, conventional liposome forming materials can be used to prepare liposomes having negative charge or neutral charge. Such materials include phosphatidyl choline, cholesterol, phosphatidylethanolamine, and the like. These materials can also advantageously be mixed with DOTMA starting materials in ratios from 0% to about 75%.

Conventional methods can be used to prepare other, noncationic liposomes. These liposomes do not fuse with cell walls as readily as cationic liposomes. However, they are taken up by macrophages in vivo, and are thus particularly effective for delivery of agents to these cells. For example, commercially dioleoylphosphatidyl choline (DOPC), dioleoylphosphatidyl glycerol (DOPG), and dioleoylphosphatidyl ethanolamine (DOPE) can be used in various combinations to make conventional liposomes, with or without the addition of cholesterol. Thus, for example, DOPG/DOPC vesicles can be prepared by drying 50 mg each of DOPG and DOPC under a stream of nitrogen gas into a sonication vial. The sample is placed under a vacuum pump overnight and is hydrated the following day with deionized water. The sample is then sonicated for 2 hours in a capped vial, using a Heat Systems model 350 sonicator equipped with an inverted cup (bath type) probe at the maximum setting while the bath is circulated at 15° C. Alternatively, negatively charged vesicles can be prepared without sonication to produce multilamellar vesicles or by extrusion through nucleopore membranes to produce unilamellar vesicles of discrete size. Other methods are known and available to those skilled in the art.

In a particularly preferred embodiment, 11β-HSD 1 protein, or nucleic acid vectors encoding 11β-HSD 1, are delivered to macrophages using liposome technology as described above. 11β-HSD 1, as described herein, catalyses the activation of inactive 11-dehydrocorticosteroids to active glucocorticoid forms thereof in vivo and in vitro in macrophages.

In a further embodiment, active glucocorticoids or inactive 11-dehydrocorticosteroid precursors are delivered to macrophages at the site of inflammation by administration of liposomes carrying such steroids.

Glucocorticoids may also be delivered systemically, preferably in inactive 11-keto or 9-α-Fluorinated forms, for reactivation by 11β-HSD enzymes in macrophages. Generally, the endogenous pool of inactive steroid is large enough to permit significant increases in active steroid concentration in macrophages which have increased levels of 11β-HSD enzymes. In a further aspect, therefore, oral and/or systemic delivery of inactive glucocorticoids is combined with the engineering of macrophages to express elevated levels of 11β-HSD 1 or 11β-HSD 2.

Engineered Macrophages

An alternative means for the upregulation of macrophage phagocytosis at sites of inflammation is to promote conversion of inactive glucocorticoids to active glucocorticoid selectively within the macrophage. To this end it has been shown that expression of 11β-HSD 1 is a characteristic of differentiation of non-phagocytic monocytes into phagocytic macrophages. Further, we have shown that inactive glucocorticoid can promote phagocytosis of apoptotic cells provided active 11β-HSD 1 is present. Therefore, driving expression of 11β-HSD enzyme (either making it expressed earlier in the differentiation of monocytes to macrophages, or to a greater degree in mature macrophages) provides a means of targeting the action of the natural excess of endogenous inactive glucocorticoid or administered inactive glucocorticoid to the macrophage.

Approaches to achieve such targeted over-expression of 11β-HSD in maturing monocytes/mature macrophages include (a) genetic engineering, which may include a macrophage specific promoter or (b) liposomal administration of a modulator that increases HSD1 expression, such as a small molecule analogue of a cytokine such as IL-4 which stimulates 11β-HSD 1 expression.

Genetically engineered macrophages may overexpress 11β-HSD enzymes as a result of the introduction of an 11β-HSD transgene, or a modulator of the endogenous 11β-HSD gene (Kluth et al., J Immunol Apr. 1, 2001;166(7):4728-36).

The engineering of macrophages to modify the activity of a modulator of glucocorticoid metabolism, such as an 11β-HSD enzyme, is carried out by conventional genetic engineering techniques. Typically this will involve transfer of a nucleic acid vector encoding the modulator in a recombinant replicable vector. The vector is used to replicate the nucleic acid in a compatible host cell. Suitable host cells include bacteria such as E. coli, eukaryotes such as yeast, mammalian cell lines and other eukaryotic cell lines, for example insect Sf9 cells.

A polynucleotide encoding a modulator according to the invention in a vector is operably linked to a control sequence that is capable of providing for the expression of the coding sequence by the host cell, i.e. the vector is an expression vector. The term “operably linked” means that the components described are in a relationship permitting them to function in their intended manner. A regulatory sequence “operably linked” to a coding sequence is ligated in such a way that expression of the coding sequence is achieved under condition compatible with the control sequences.

The control sequences can be modified, for example by the addition of further transcriptional regulatory elements to make the level of transcription directed by the control sequences more responsive to transcriptional modulators.

Vectors of the invention can be transformed or transfected into macrophages to provide for expression of the modulator therein.

The vectors can be for example, plasmid or virus vectors provided with an origin of replication, optionally a promoter for the expression of the said polynucleotide and optionally a regulator of the promoter. The vectors can contain one or more selectable marker genes, for example a neomycin resistance gene.

Control sequences operably linked to sequences encoding the protein of the invention include promoters/enhancers and other expression regulation signals. These control sequences can be selected to be compatible with the host cell for which the expression vector is designed to be used in. The term “promoter” is well-known in the art and encompasses nucleic acid regions ranging in size and complexity from minimal promoters to promoters including upstream elements and enhancers.

The promoter is typically selected from promoters which are functional in mammalian cells, although prokaryotic promoters and promoters functional in other eukaryotic cells can be used. The promoter is typically derived from promoter sequences of viral or eukaryotic genes. For example, it can be a promoter derived from the macrophage, such as the CD68 promoter [Adenovirus-mediated gene transfer of a secreted form of human macrophage scavenger receptor inhibits modified low-density lipoprotein degradation and foam-cell formation in macrophages. Laukkanen J, Lehtolainen P, Gough P J, Greaves D R, Gordon S, Yla-Herttuala S. CIRCULATION 101: (10) 1091-1096 (2000)] (so that 11βHSD1 expression is directed in circulating monocytes and their progeny) or the Mouse macrophage metalloelastase (MME) promoter [Induction and regulation of macrophage metalloelastase by hyaluronan fragments in mouse macrophages. Horton M R, Shapiro S, Bao C, Lowenstein C J, Noble P W. JOURNAL OF IMMUNOLOGY 162: (7) 4171-4176 Apr. 1, 1999] (so that 11βHSD1 expression is activated as monocytes move into inflamed sites). The promoter can be a promoter that functions in a ubiquitous manner (such as promoters of α-actin, β-actin, tubulin) or, alternatively, a tissue-specific manner. Tissue-specific promoters specific for macrophages are particularly preferred. They can also be promoters that respond to specific stimuli, for example promoters that bind steroid hormone receptors. Viral promoters can also be used, for example the Moloney murine leukaemia virus long terminal repeat (MMLV LTR) promoter, the Rous sarcoma virus (RSV) LTR promoter or the human cytomegalovirus (CMV) IE promoter.

It can also be advantageous for the promoters to be inducible so that the levels of expression of the heterologous gene can be regulated during the life-time of the cell. Inducible means that the levels of expression obtained using the promoter can be regulated.

In addition, any of these promoters can be modified by the addition of further regulatory sequences, for example enhancer sequences. Chimeric promoters can also be used comprising sequence elements from two or more different promoters described above.

Techniques for transformation of macrophages are known in the art, and include DNA transfection techniques such as electroporation or lipofection and viral transduction. Uptake of naked nucleic acid constructs by mammalian cells is enhanced by several known transfection techniques for example those including the use of transfection agents. Example of these agents include cationic agents (for example calcium phosphate and DEAE-dextran) and lipofectants (for example lipofectam™ and transfectam™). Typically, nucleic acid constructs are mixed with the transfection agent to produce a composition.

In a preferred embodiment, peripheral blood mononuclear cells are isolated from human peripheral blood at laboratory scale by standard techniques procedures (Sandlie and Michaelsen, 1996, in Antibody engineering: a practical approach. Ed McCafferty et al. Chapter 9) and at large scale by elutriation (eg using Ceprate from CellPro). Adherent cells (essentially monocytes) are enriched by adherence to plastic overnight and cells are allowed to differentiate along the macrophage differentiation pathway by culturing adherent cells for 1-3 weeks.

Monocytes and macrophages are transfected with an expression vector capable of expressing 11β-HSD 1 in human cells. For constitutive high level expression, 11β-HSD 1 is expressed in a vector which utilises the hCMV-MIE promoter-enhancer, pCI (Promega).

A variety of transfection methods can be used to introduce vectors into monocytes and macrophages, including particle-mediated DNA delivery (biolistics), electroporation, cationic agent-mediated transfection (eg using Superfect, Qiagen). Each of these methods is carried out according to the manufacturer's instructions, taking into account the parameters to be varied to achieve optimal results as specified by the individual manufacturer. Alternatively, viral vectors may be used such as defective Adenovirus vectors (Microbix Inc or Quantum Biotechnologies Inc).

In the case of the need for treatment of a chronic condition, haematopoietic stem cells which give rise to the monocyte/macrophage lineage may be transformed with nucleic acids encoding 11β-HSD 1, thus providing a stable source of 11β-HSD 1 enhanced macrophage cells.

Engineered macrophages may be administered to the site of inflammation by topical or systemic delivery. For example, macrophages may be administered directly to an inflamed joint, lung or peritoneum. Macrophages injected at the site of inflammation are capable of homing to inflamed tissues (Kluth et al., J Immunol Apr. 1, 2001; 166(7):4728-36).

Formulation of Pharmaceutical Compositions

A pharmaceutical composition according to the invention is a composition of matter comprising an active or inactive steroid as active ingredient. The active ingredients of a pharmaceutical composition comprising the combination according to the invention exhibit excellent therapeutic activity in the successful resolution of inflammatory responses. Dosage regima can be adjusted to provide the optimum therapeutic response. For example, several divided doses can be administered daily or the dose can be proportionally reduced as indicated by the exigencies of the therapeutic situation.

The active compound can be administered in a convenient manner such as by the topical, oral, intravenous, intramuscular, subcutaneous, intranasal, intradermal or suppository routes or implanting (e.g. using slow release molecules). Depending on the route of administration, the active ingredient can be required to be coated in a material to protect said ingredients from the action of enzymes, acids and other natural conditions which can inactivate said ingredient.

A topical formulation can be liposome-based. Liposomes include water-in-oil-in-water CGF emulsions as well as conventional liposomes, as described in further detail above.

The active compound can also be administered parenterally or intraperitoneally. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as lecithin, by the maintenance of the required particle size in the case of dispersion and by the use of superfactants.

The prevention of the action of microorganisms can be brought about by various antibacterial and antifungal agents, for example, parabens, chlorobutanol, phenol, sorbic acid, thirmerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compound in the required amount in the appropriate solvent with various of the other ingredients enumerated above, as required, followed by filtered sterilisation. Generally, dispersions are prepared by incorporating the sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

Tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavouring agent such as peppermint, oil of wintergreen, or cherry flavouring. When the dosage unit form is a capsule, it can contain, in addition to materials of the above type, a liquid carrier.

Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound can be incorporated into sustained-release preparations and formulations.

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in unit dosage form for ease of administration and uniformity of dosage. Unit dosage form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel unit dosage forms of the invention are dictated by and directly dependent on (a) the unique characteristics of the active material and the particular therapeutic effect to be achieved, and (b) the limitations inherent in the art of compounding such as active material for the treatment of disease in living subjects having a disease condition in which bodily health is impaired.

The principal active ingredients are compounded for convenient and effective administration in effective amounts with a suitable pharmaceutically acceptable carrier in dosage unit form. In the case of compositions containing supplementary active ingredients, the dosages are determined by reference to the usual dose and manner of administration of the said ingredients.

In a further aspect there is provided the combination of the invention as hereinbefore defined for use in the treatment of disease which involves an inflammatory response, such as peritonitis.

The invention is further described below, with reference to the following non-limiting examples.

### EXAMPLES

Methods

Bone Marrow Derived Macrophages (BMD Mφ)—bone marrow was extracted from femurs (C57BI/6 mice) and cultured @4×10⁵ cells/ml in DMEM/10% FCS/10% L929 cell line supernatant for 8 days.

Thioglycollate Elicited Peritoneal Mφ (TPM Mφ)—peritoneal inflammatory Mφ were ravaged from C57BI/6 mice 4 days after intra-peritoneal injection of 4% thioglycollate solution. Cells were adhered @1×10⁶ cells/ml, washed and cultured 24 hrs in DMEM/10% FCS.

Human Monocytes—monocytes isolated from peripheral blood by dextran sedimentation and percoll gradient methods. Monocytes were differentiated to Mφ @4×10⁶ cells/ml over 4 days in Iscoves/10% autologous serum.

### Example 1

Phagocytosis of Apoptotic Neutrophils by Macrophages

Culture of neutrophils (PMN) under appropriate conditions leads to apoptosis and ingestion of apoptotic cells by macrophages, leading to non-necrotic clearing of neutrophil matter. Failure to clear such apoptotic neutrophil material in a timely manner leads to deleterious chronic inflammation and ongoing tissue damage.

Fresh PMN were isolated from peripheral blood and aged to apoptosis in Teflon wells over 24 hrs in Iscoves/10% autologous serum. Mφ were treated with glucocorticoids as described in the figures. Mφ and aPMN were washed with 1×PBS and interacted in serum free Iscoves for 30 mins at 37° C. The cell layer was gently washed to remove adherent cells, fixed in 1×Formalin and stained with PMN selective myeloperoxidase.

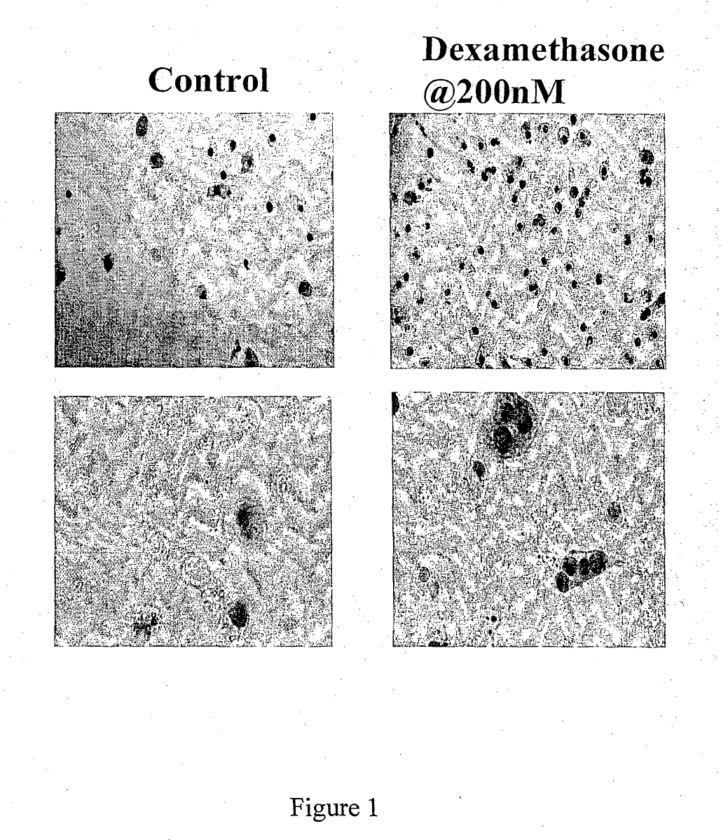

Using 200 nM dexamethasone, we observed a marked increase in the uptake of apoptotic neutrophils by macrophages. This is shown in FIG. 1. Glucocorticoid action increases both the numbers of macrophages involved in phagocytosis and the number of PMN ingested per macrophage.

### Example 2

Expression of 11β-HSD1 in Macrophages

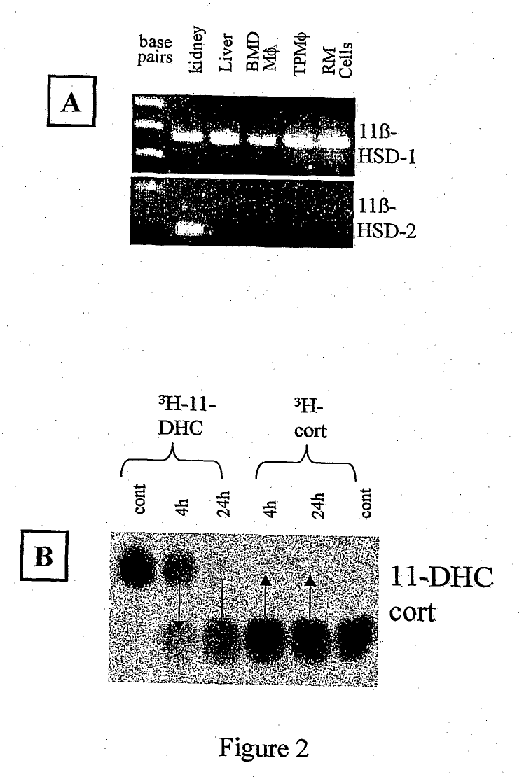

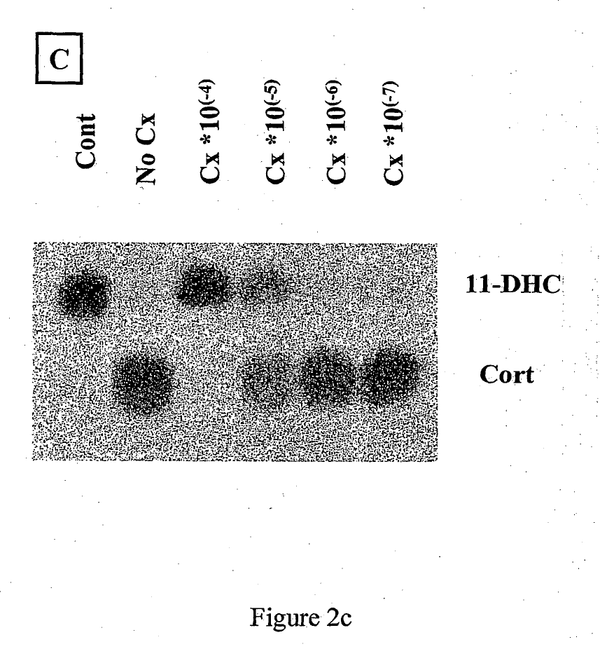

FIG. 2a shows an RT-PCR of transcripts obtained from murine kidney, liver and macrophages. The expression of 11β-HSD1 in macrophages can clearly be observed, as can the absence of 11β-HSD2 expression.

### Example 3

11β-HSD1 converts inactive 11-dehydrocorticosteroid to active glucocorticoid ³H-Corticosterone (cort) or ³H-11-dehydrocorticosterone (11 DHC) were added to Mφ cultures at a concentration of 0.25 μCi/ml. Samples were taken at different times and ³H-cort and ³H-11-DHC were separated from media samples by thin layer chromatography and analysed by phosphorimaging.

In FIG. 2b, the results of an experiment are shown in which the conversion of inactive 11-dehydrocorticosterone (A) to active glucocorticoid corticosterone (B) is monitored in mouse. 11β-HSD1 clearly shows conversion of (A) to (B) over a 24 hour period. However, as shown in FIG. 2c, this effect can be blocked by high concentrations of the 11β-HSD1 inhibitor carbenoxolone.

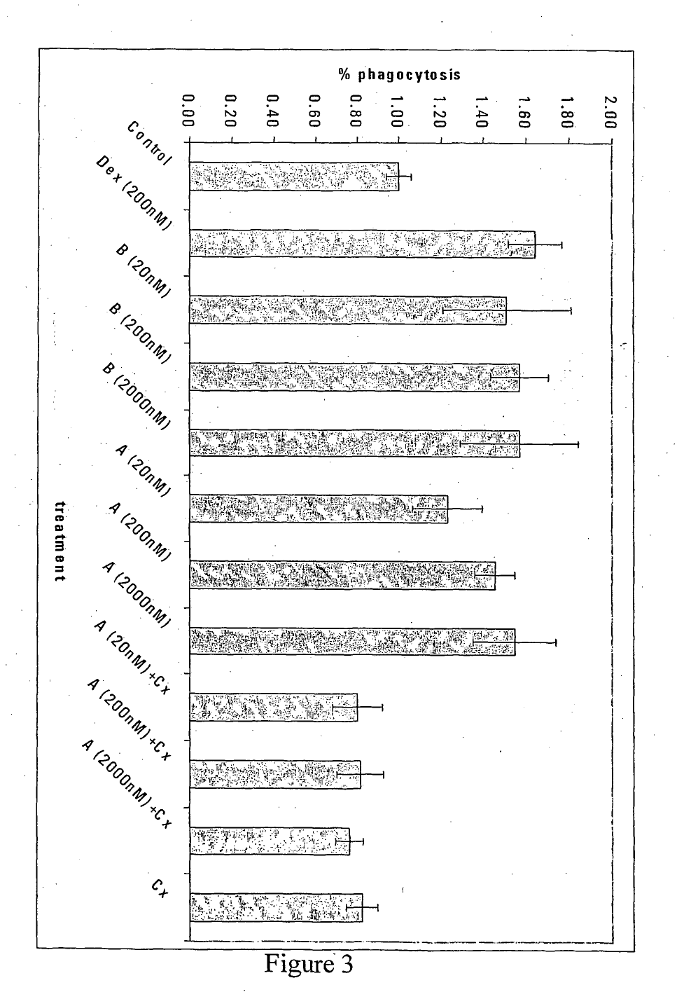

The effect is also observed in phagocytosis by murine peripheral macrophages, as shown in FIG. 3. Active glucocorticoid raises the phagocytosis performance of macrophages two-fold over 24 hours, as does inactive 11-dehydrocorticosterone after incubation (to allow its activation to corticosterone by 11β-HSD1). However, incubation of macrophages with inactive 11-dehydrocorticosterone in the presence of the 11β-HSD1 inhibitor carbenoxolone completely abolishes the effects thereof.

### Example 4

Phagocytosis in 11β-HSD^{-/-} Macrophages

Transgenic 11β-HSD^{-/-} mice have been described previously (Kotelevstev et al., PNAS (USA) 94:14924-14929). Macrophages are isolated from control and 11β-HSD^{-/-} mice and assayed in a PMN phagocytosis assay as described above.

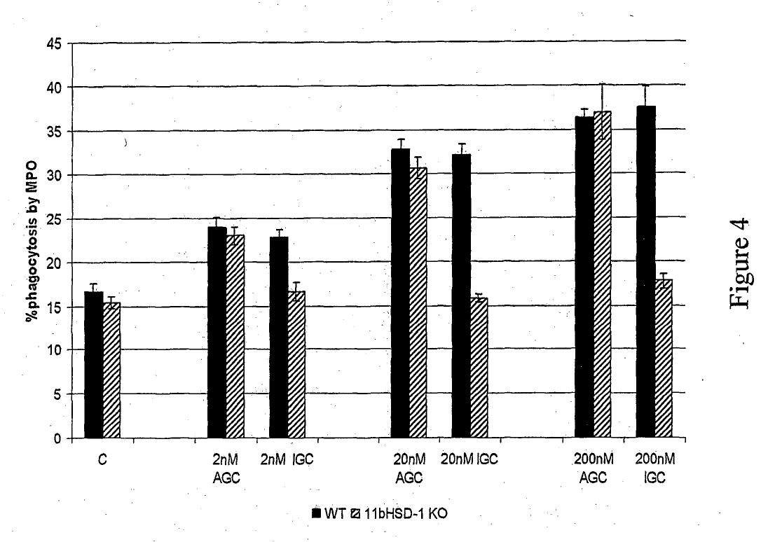

As shown in FIG. 4, in vitro enhancement of phagocytosis of apoptotic polymorphonuclear cells (PMN) by murine BMD Mφ after 48 hrs treatment with ‘active’ (AGC=corticosterone) or ‘inactive’ (IGC=11-DHC) 11-dehydrocorticosteroid (n=4/group).

The enhanced phagocytic effect conferred by 11-DHC (IGC) is abrogated in 11βHSD-1^{-/-} Mφ. In contrast loss of 11βHSD1 has no effect upon active GC (cort) action.

### Example 5

Induction of 11β-HSD 1 Expression In Vivo and In Vitro.

Peritonitis can be induced in vivo in mice by injection of thioglycollate (1 ml 3% thioglycollate, sterile solution) intraperitoneally. By analysis of peritoneal macrophages, we have shown that 11β-HSD 1 is upregulated rapidly upon inflammation of the peritoneum.

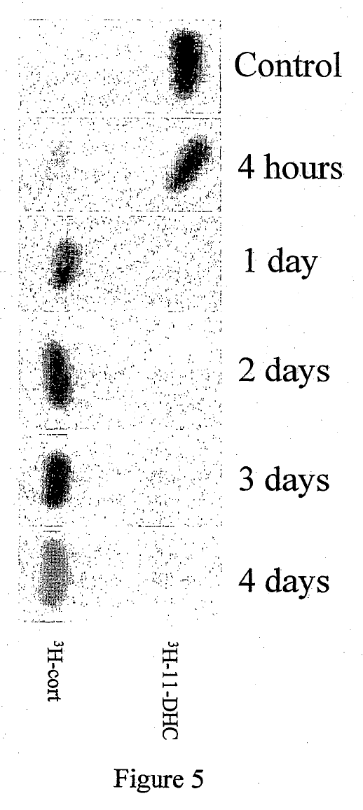

FIG. 5 shows that conversion of inert 11-DHC to active corticosterone (cort) by peritoneal macrophages is minimal before peritonitis (control), but is rapidly induced (within less than 4 h), peaking 2-3 days after onset. These data imply that 11β-HSD 1 expression is upregulated in vivo in response to inflammation.

### Example 6

Glucocorticoid Activation is Influenced by Macrophage Differentiation

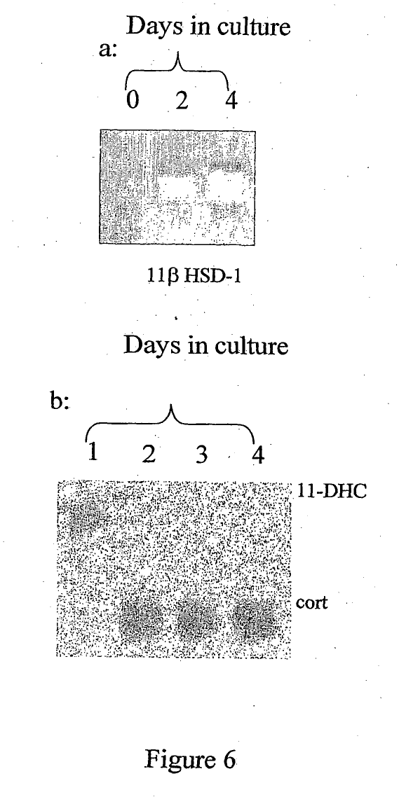

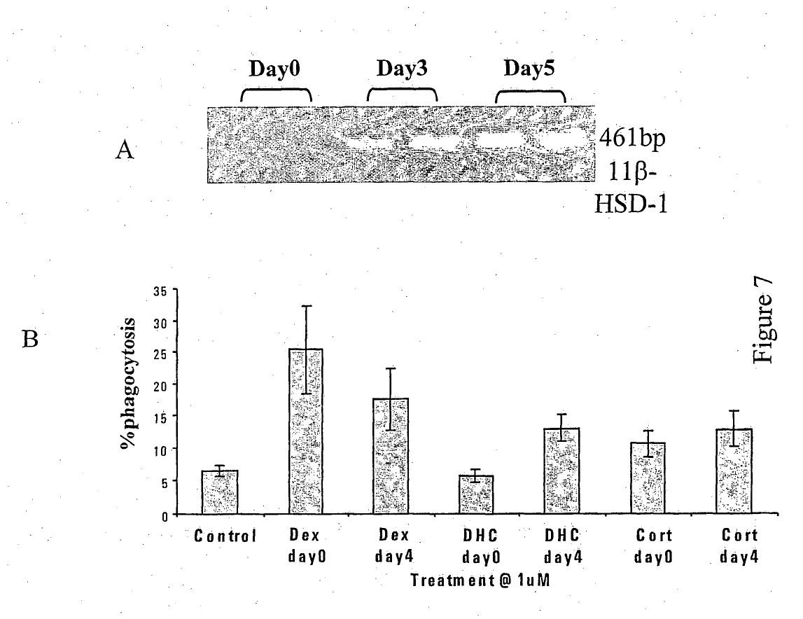

The activation of inactive glucocorticoid correlates with the production of 11β-HSD1 in macrophages during macrophage differentiation in cell culture, as shown in FIGS. 6-8. In FIG. 6a, RT-PCR of monocyte/macrophage differentiating cultures shows that 11βHSD-1 mRNA is absent in day 0 human monocytes, but is upregulated by day 2. In FIG. 6b, an 11β-reductase enzyme assay shows that 11βHSD-1 activity increases during the first 3 days of culture (24 h incubation with ³H-11-DHC, as above). Similar results are shown in FIG. 7, where macrophage phagocytosis is measured.

Differentiating monocytes were obtained and studied by analysing phagocytosis levels (FIG. 7) and 11-DHC conversion to corticosterone (FIG. 6). In both cases, conversion of 11-DHC to corticosterone is enhanced during macrophage differentiation, in synchrony with increase in 11β-HSD 1 expression.

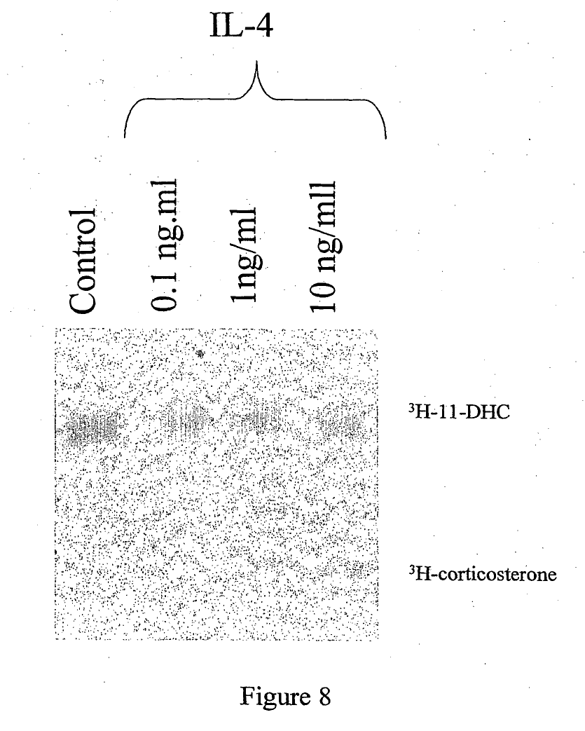

The manner in which differentiating macrophages acquire 11β-HSD-1 activity can be influenced by cytokines. In the experiment shown in FIG. 8, human monocytes at the initiation of differentiation into macrophages were incubated with a range of doses of IL-4. 11β-reductase activity was measured by incubation with tritiated 11-DHC. Administration of IL-4 dose-relatedly increased conversion of inert 11-DHC to active corticosterone.

### Example 7

11β-HSD 1 Activity Reduces After Resolution of Inflammation

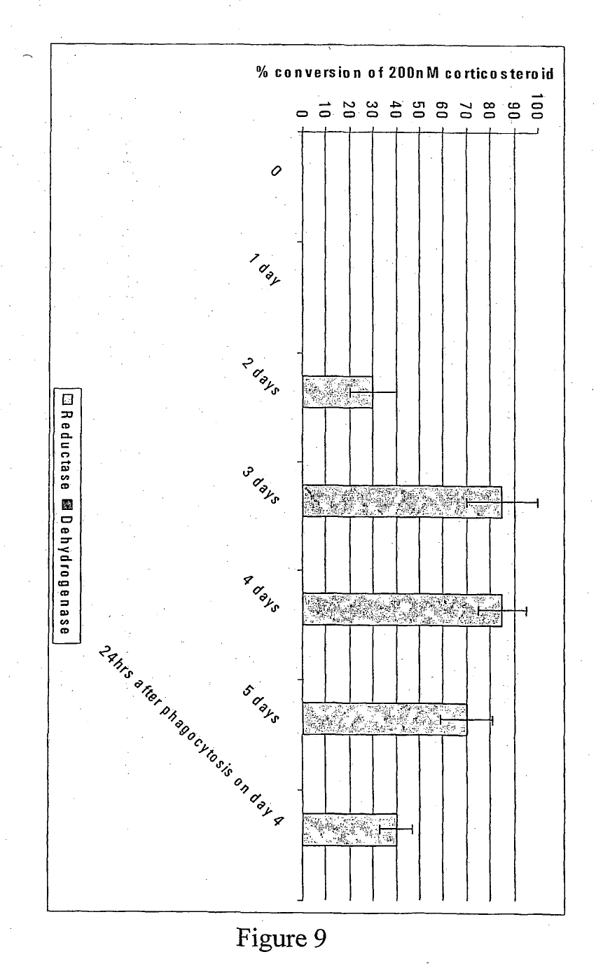

11β-HSD1 activity is purely reductive in human macrophages and is induced during monocyte to macrophage differentiation. As shown in FIG. 9, enzyme activity falls after phagocytosis suggesting that amplification of glucocorticoid action is reduced after macrophages have functioned to resolve inflammation.

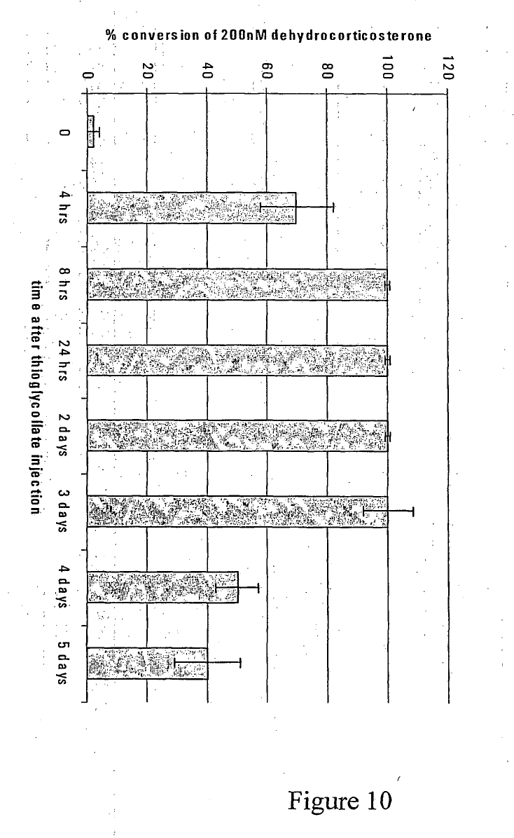

The same is confirmed in vivo. In FIG. 10, 11β-HSD1 reductase is induced in mouse peritoneal macrophages during the development of thioglycollate-induced peritonitis. As in human macrophages in vitro, in mouse peritoneal macrophage 11β-HSD1 reductase activity falls after the peak of phagocytosis, suggesting that amplification of glucocorticoid action is reduced after macrophages have functioned to resolve the inflammation.

In both cases, no dehydrogenase activity is detected, confirming that 11β-HSD 1 functions exclusively as a reductase in macrophages.

### Example 8

Glucocorticoids Potentiate Macrophage Phagocytosis In Vivo

Neutrophils prepared from normal blood were cultured overnight in order to undergo apoptosis. The apoptotic cells were labelled with fluorescent tracking dye. They were then injected into the peritoneum of a mouse which had an ongoing peritoneal inflammatory response induced by injection of thioglycollate four days before. At the stage of injection of the apoptotic cells, the majority of the inflammatory cells in the peritoneum are macrophages.

After 30 minutes interaction the mouse is killed and the peritoneal cells harvested and examined by microscopy. Under base line conditions using administration of 5,000,000 neutrophils, 24.4 plus or minus 8.3% (mean plus or minus SD; N=24) of macrophages contained fluorescent apoptotic cells.

When the mouse had been treated with a dose of dexamethasone calculated to give a final concentration of 200 nmol in the whole mouse, there was an increase in phagocytosis of administered apoptotic cells to 47.4 plus or minus 11.1% of macrophages. This was significant at the P less than 0.01 level by ANOVA. The glucocorticoid was administered intraperitoneally.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

The invention will now be further described by the following numbered paragraphs:

1. Use of a modulator of glucocorticoid metabolism in the manufacture of a composition for the potentiation of a successful resolution of an inflammatory response in a mammal.

2. Use according to paragraph 1, wherein the modulator increases the intracellular concentration of glucocorticoids in macrophages.

3. Use according to paragraph 1 or paragraph 2, wherein the modulator is a modulator of the activity of an 11b-HSD1 reductase enzyme.

4. An engineered macrophage having increased endogenous biosynthesis of active glucocorticoid.

5. A macrophage according to paragraph 4 which is genetically engineered.

6. A genetically engineered macrophage according to paragraph 5, wherein endogenous 11b-HSD1 activity is upregulated.

7. A macrophage according to any one of paragraphs 4 to 6, for use in the potentiation of a successful resolution of the inflammatory response in a mammal.

8. Use of a glucocorticoid or 11-dehydrocorticosteroid in the manufacture of a composition for the potentiation of a successful resolution of the inflammatory response in a mammal.

9. Use according to paragraph 8, wherein the 11-dehydrocorticosteroid is activated by 11b-HSD1.

10. Use according to paragraph 8 or paragraph 9, wherein the glucocorticoid is administered in an inactive form.

11. Use according to paragraph 10, wherein the inactive precursor of the glucocorticoid is a 11-dehydroxycorticosteroid.

12. Use according to any one of paragraphs 8 to 11, wherein the composition further comprises a modulator of glucocorticoid metabolism according to any one of paragraphs 1 to 3.

13. A method of potentiating a successful resolution of the inflammatory response in a mammal, comprising administering to a mammal in need thereof a composition comprising a glucocorticoid or 11-dehydrocorticosteroid.

14. A method according to paragraph 13, wherein the 11-dehydrocorticosteroid is activated by 11b-HSD1.

15. A method according to paragraph 13 or paragraph 14, wherein the glucocorticoid is administered in an inactive form.

16. A method according to paragraph 15, wherein the inactive precursor of the glucocorticoid is a 11-dehydroxycorticosteroid.

17. A method according to any one of paragraphs 13 to 16, wherein the composition further comprises a modulator of glucocorticoid metabolism according to any one of paragraphs 1 to 3.

18. A pharmaceutical composition comprising a glucocorticoid in inactive form.

19. A pharmaceutical composition according to paragraph 18, wherein the inactive precursor of the glucocorticoid is a 11-dehydroxycorticosteroid.

20. A pharmaceutical composition according to paragraph 18 or paragraph 19, wherein the 11-dehydrocorticosteroid is activated by 11 b-HSD1.

21. A pharmaceutical composition according to any one of paragraphs 18 to 20, wherein the composition further comprises a modulator of glucocorticoid metabolism according to any one of paragraphs 1 to 3.

1. A method of using a modulator of glucocorticoid metabolism in the manufacture of a composition for the potentiation of a successful resolution of an inflammatory response in a mammal.

2. The method of claim 1, wherein the modulator increases the intracellular concentration of glucocorticoids in macrophages.

3. The method of claim 1, wherein the modulator is a modulator of the activity of an 11b-HSD1 reductase enzyme.

4. An engineered macrophage having increased endogenous biosynthesis of active glucocorticoid.

5. The macrophage of claim 4, wherein the macrophage is genetically engineered.

6. The genetically engineered macrophage of claim 5, wherein endogenous 11b-HSD1 activity is upregulated.

7. The macrophage of claim 4, for use in the potentiation of a successful resolution of the inflammatory response in a mammal.

8. A method of using a glucocorticoid or 11-dehydrocorticosteroid in the manufacture of a composition for the potentiation of a successful resolution of the inflammatory response in a mammal.

9. The method of claim 8, wherein the 11-dehydrocorticosteroid is activated by 11b-HSD1.

10. The method of claim 8, wherein the glucocorticoid is administered in an inactive form.

11. The method of claim 10, wherein the inactive precursor of the glucocorticoid is a 11-dehydroxycorticosteroid.

12. A method of using a glucocorticoid or 11-dehydrocorticosteroid in the manufacture of a composition for the potentiation of a successful resolution of the inflammatory response in a mammal, wherein the composition further comprises a modulator of glucocorticoid metabolism according to claim 1.

13. A method of potentiating a successful resolution of the inflammatory response in a mammal, comprising administering to a mammal in need thereof a composition comprising a glucocorticoid or 11-dehydrocorticosteroid.

14. The method of claim 13, wherein the 11-dehydrocorticosteroid is activated by 11b-HSD1.

15. The method of claim 13, wherein the glucocorticoid is administered in an inactive form.

16. The method of claim 15, wherein the inactive precursor of the glucocorticoid is a 11-dehydroxycorticosteroid.

17. A method of potentiating a successful resolution of the inflammatory response in a mammal, comprising administering to a mammal in need thereof a composition comprising a glucocorticoid or 11-dehydrocorticosteroid, wherein the composition further comprises a modulator of glucocorticoid metabolism according to claim 1.

18. A pharmaceutical composition comprising a glucocorticoid in inactive form.

19. The pharmaceutical composition of claim 18, wherein the inactive precursor of the glucocorticoid is a 11-dehydroxycorticosteroid.

20. The pharmaceutical composition of claim 18, wherein the 11-dehydrocorticosteroid is activated by 11b-HSD1.

21. A pharmaceutical composition comprising a glucocorticoid in inactive form, wherein the composition further comprises a modulator of glucocorticoid metabolism according to claim 1.
